(19) United States
(12) Patent Application Publication
(10) Pub. No.: US 2014/0228680 A1
(43) Pub. Date: Aug. 14, 2014

(54) GUIDING-TYPE MEDICAL SYSTEM

(71) Applicant: OLYMPUS CORPORATION, Tokyo (JP)

(72) Inventor: Hiroshi FUKUDA, Tokyo (JP)

(73) Assignee: OLYMPUS CORPORATION, Tokyo (JP)

(21) Appl. No.: 14/257,049

(22) Filed: Apr. 21, 2014

Related U.S. Application Data
(63) Continuation of application No. PCT/JP2012/081488, filed on Dec. 5, 2012.

(30) Foreign Application Priority Data
Dec. 9, 2011 (JP) 2011-270258

(51) Int. Cl. A61B 8/08; A61M 25/01

(52) U.S. Cl. CPC A61B 8/0841; A61M 25/0127
USPC 600/424

(57) ABSTRACT

A medical appliance is easily inserted into a desired blood vessel route. There is provided a guiding-type medical system, including: a cylindrical or linear guidewire made of an elastic body that can be inserted into a blood vessel; and a guiding device having a device head portion that can be inserted into a body to guide the guidewire inserted into the blood vessel. The device head portion includes: an ultrasonic array element that can perform an ultrasound scan along one plane to acquire an ultrasound image; and a permanent magnet linearly movable in a direction along the one plane. At a distal end of the guidewire, a magnetic head on which magnetic attraction force induced by the permanent magnet is made to act is provided.

### TECHNICAL FIELD

The present invention relates to a guiding-type medical system.

### BACKGROUND ART

There has conventionally been known a guiding-type medical system in which medical appliances, such as guidewires and catheters that are inserted into blood vessels, are guided to a desired blood vessel route at a blood vessel branch point (see, for example, PTL 1 and PTL 2). In a guiding-type medical system disclosed in PTL 1, a powerful magnet is placed outside the body of a patient to generate a field gradient inside the body. The generated field gradient guides a catheter with a magnet mounted therein into a blood vessel and/or the heart.

### CITATION LIST

### Patent Literature

{PTL 1}

U.S. Pat. No. 6,975,197

{PTL 2}

Japanese Unexamined Patent Application, Publication No. 2005-161052

### SUMMARY OF INVENTION

### Technical Problem

However, the guiding-type medical systems disclosed in PTLs 1 and 2 have a powerful magnet, and therefore apparatus bodies become large and expensive.

An object of the present invention is to provide a guiding-type medical system that enables a medical appliance to be easily inserted to a desired blood vessel route.

### Solution to Problem

In order to accomplish the above object, the present invention provides the following solution.

One aspect of the present invention provides a guiding-type medical system, including: a cylindrical or linear medical appliance made of an elastic body that can be inserted into a blood vessel; and a guiding device having an insertion portion that can be inserted into a body to guide the medical appliance inserted into the blood vessel, wherein the insertion portion includes: an ultrasound probe that can perform an ultrasound scan along one plane to acquire an ultrasound image; and a magnet linearly movable in a direction along the one plane, and at a distal end of the medical appliance, a magnetic member made of a magnetic material on which magnetic attraction force induced by the magnet is made to act is provided.

### BRIEF DESCRIPTION OF DRAWINGS

{FIG. 1}

FIG. 1 is a schematic view illustrating a guiding-type medical system according to a first embodiment of the present invention and a region in the vicinity of the heart of a patient.

{FIG. 2}

FIG. 2 is a schematic view illustrating a guidewire of FIG. 1.

{FIG. 3}

FIG. 3 is a general view of the guiding device of FIG. 1.

{FIG. 4}

FIG. 4(a) illustrates a shaft portion of FIG. 3 in the state of linearly extending, and FIG. 4(b) illustrates the shaft portion in the state of being curved by the operation of a curving mechanism.

{FIG. 5}

FIG. 5(a) is a cross sectional view of a device head portion of FIG. 3, FIG. 5(b) is a cross sectional view of the device head portion of FIG. 5(a) cut at another position, and FIG. 5(c) illustrates a magnet moving mechanism.

{FIG. 6}

FIG. 6 illustrates the relation between a region around a blood vessel branch part and an ultrasound scanning position.

{FIG. 7}

FIG. 7(a) illustrates an ultrasound image obtained by performing a scan at an L-L′ position in FIG. 6, FIG. 7(b) illustrates an ultrasound image obtained by performing a scan at an M-M′ position in FIG. 6, and FIG. 7(c) illustrates an ultrasound image obtained by performing a scan at an N-N′ position in FIG. 6.

{FIG. 8}

FIG. 8 illustrates an ultrasound image displayed on a monitor when a scan is performed at the M-M′ position in FIG. 6.

{FIG. 9}

FIG. 9 illustrates the guidewire being moved toward a blood vessel branch part.

{FIG. 10}

FIG. 10(a) illustrates a magnetic head of the guidewire being attracted to the magnet at the blood vessel branch part, and FIG. 10(b) illustrates an ultrasound image acquired in the case of FIG. 10(a).

{FIG. 11}

FIG. 11(a) illustrates the magnetic head of the guidewire being attracted to the magnet of the guiding device so as to be moved, and FIG. 11(b) illustrates an ultrasound image acquired in the case of FIG. 11(a).

{FIG. 12}

FIG. 12(a) illustrates the magnetic head of the guidewire being attracted to the magnet of the guiding device so as to be further moved, and FIG. 12(b) illustrates an ultrasound image acquired in the case of FIG. 12(a).

{FIG. 13}

FIG. 13 is a cross sectional view of a guidewire of a guiding-type medical system according to a first modified example of the first embodiment of the present invention.

{FIG. 14}

FIG. 14 is a cross sectional view of a device head portion of a guiding-type medical system according to a second modified example of the first embodiment of the present invention.

{FIG. 15}

FIG. 15(a) is a cross sectional view of a device head portion of a guiding-type medical system according to a second embodiment of the present invention, and FIG. 15(b) is a cross sectional view illustrating a magnet being moved from the state of FIG. 15(a).

{FIG. 16}

FIG. 16(a) is a cross sectional view of a device head portion of a guiding-type medical system according to a third embodiment of the present invention, FIG. 16(b) is a cross sectional view of the device head portion viewed in an axis direction, and FIG. 16C is a cross sectional view illustrating the device head portion of FIG. 16(a) cut at another position.

{FIG. 17}

FIG. 17(a) illustrates a range of ultrasound irradiation by two ultrasonic array elements of FIG. 16(a), and FIG. 17(b) illustrates a range of ultrasound irradiation by only one ultrasonic array element placed on one side of the magnet as a reference example of FIG. 17(a).

### DESCRIPTION OF EMBODIMENTS

### First Embodiment

A guiding-type medical system according to the first embodiment of the present invention is described hereinbelow with reference to the accompanying drawings.

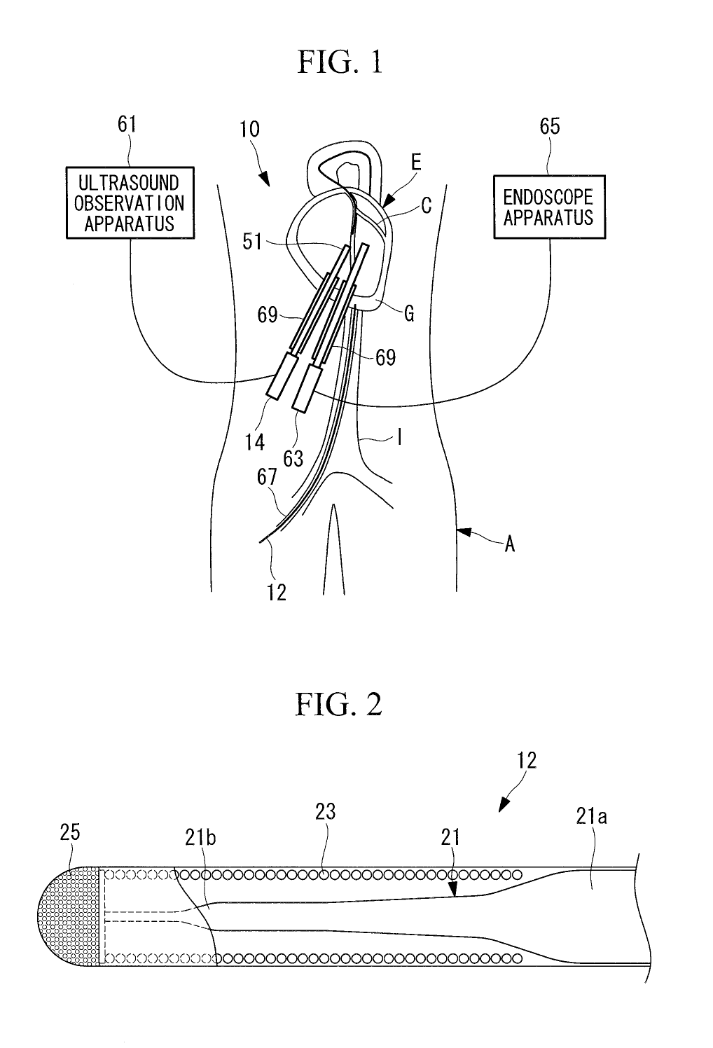

As illustrated in FIG. 1, a guiding-type medical system 10 according to the present embodiment includes a medical appliance 12, such as a cylindrical or linear guidewire and/or catheter, inserted into a coronary artery C of a patient A, and a guiding device 14 having a device head portion (insertion portion) 51 that can be inserted into the body of the patient A to guide the medical appliance 12 inserted into the coronary artery C. Hereinafter, a guidewire is described as an example of the medical appliance 12.

The guiding-type medical system 10 is connected to an ultrasound observation apparatus 61, so that an acquired ultrasound image can be displayed on a monitor 61a of the ultrasound observation apparatus 61. In FIG. 1, a heart E and a pericardial cavity G are shown. There are also shown an endoscope 63, an endoscope apparatus 65 connected to the endoscope 63, an introducer 67 which can insert the guidewire 12 to a main artery I, and sheaths 69 with a steering mechanism (e.g., Steerable Introducer Agilis made by St. Jude Medical, Inc.) into which the guiding device 14 and the endoscope 63 can respectively be inserted.

As illustrated in FIG. 2, the guidewire 12 includes an generally columnar shaft 21 extending in the axis direction, a coil 23 placed so as to cover the circumference of the shaft 21, and a magnetic head (magnetic member) 25 provided at a distal end of the shaft 21.

The shaft 21 has a base end 21a having an outside diameter size generally identical to an outside diameter size of the magnetic head 25, and a tapered portion 21b having a tapered form extending from the base end 21a toward the distal end. Since the diameter size of the shaft 21 is gradually decreased toward the distal end with the tapered portion 21b, the distal end of the guidewire 12 is easily bendable.

The coil 23 is placed around the tapered portion 21b of the shaft 21 at intervals in a radial direction.

The magnetic head 25 is made of a cannonball-shaped or hemispherical magnetic material which projects toward the distal end of the guidewire 12. The magnetic head 25 is shaped to have a plurality of minute projections and indentations on the outer surface thereof.

The circumferences of the shaft 21, the coil 23, and the magnetic head 25 are coated with a hydrophilic coating layer (illustration omitted). This reduces frictional force generated when the guidewire 12 comes into contact with the introducer 67 or intravascular walls, and facilitates backward and forward movement of the guidewire 12. The outer surface of the magnetic head 25 which is formed to have projections and indentations are also coated with a coating layer so as to generally be smoothed.

The thus-configured guidewire 12 can be magnetized in a magnetic field by the magnetic head 25 provided at the distal end, while retaining an original mechanism of the guidewire.

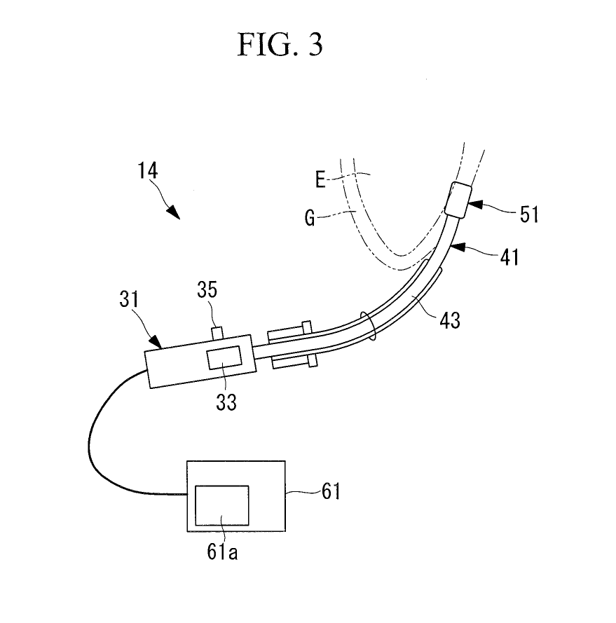

As illustrated in FIG. 3, the guiding device 14 includes a grip portion 31 gripped by an operator, and a shaft portion 41 extending from the grip portion 31 toward the distal end so as to be connected to the device head portion 51.

The grip portion 31 includes a lever 33 that operates the shaft portion 41 and the device head portion 51, and a magnet drive knob 35.

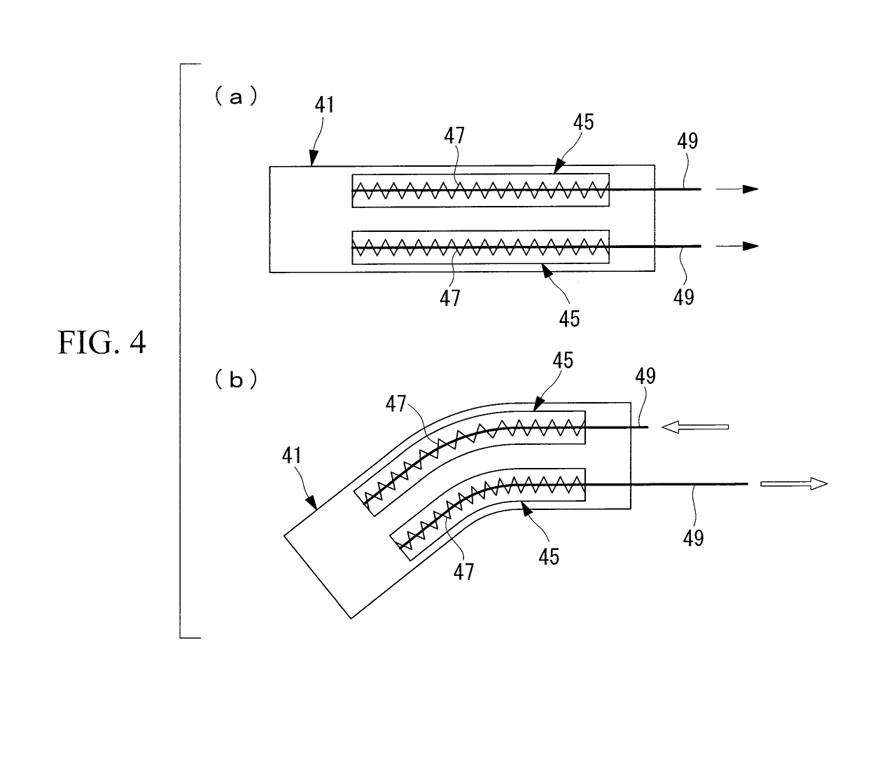

The shaft portion 41 includes a shaft main body 43 and a curving mechanism 45 that curves the shaft main body 43 in a direction intersecting the axis direction as illustrated in FIGS. 4(a) and 4(b).

The shaft main body 43 is pre-shaped so that the surface of the device head portion 51 that comes into contact with a cardiac muscle tissue in the pericardial cavity G is gradually curved toward the direction of the heart E. This makes it possible to bring the device head portion 51 into close contact with the surface of the heart E without the need of a vertical steering mechanism.

The curving mechanism 45, which is housed in the shaft main body 43, includes two steering coils 47 and steering wires 49.

Both sides of each of these two steering coils 47 are fixed to the shaft main body 43 with a margin of expanding both the sides. The steering coils 47 are respectively connected to the lever 33 of the grip portion 31 through the respective steering wires 49.

In the curving mechanism 45, if one steering wire 49 is pulled and the other steering wire 49 is loosened in response to operation of the lever 33, the steering coil 47 connected to the pulled steering wire 49 is contracted, while the steering coil 47 connected to the other steering wire 49 is extended. As a consequence, the shaft main body 43 can be changed from a linearly extending state as illustrated in FIG. 4(a) to a state curved in one direction as illustrated in FIG. 4(b). This allows curving operation to curve the shaft 21 in a horizontal direction with respect to the surface of the heart E.

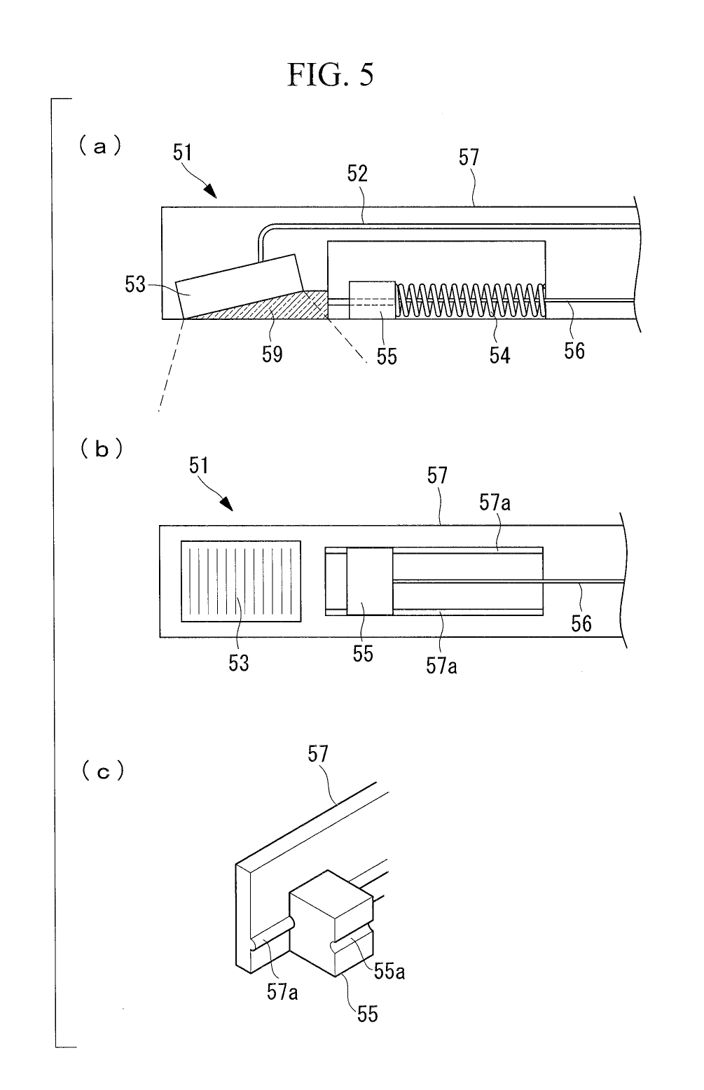

As illustrated in FIGS. 5(a) and 5(b), the device head portion 51 includes an ultrasonic array element (ultrasound probe) 53 that can perform an ultrasound scan along one plane to acquire an ultrasound image. The device head portion 51 also includes a permanent magnet (magnet) 55 that can linearly move in a direction along the one plane, and a case 57 that houses these component members.

The ultrasonic array element 53 is placed at the distal end of the case 57 with an inclination so that an ultrasound emitting surface is made to face in the radial direction of the case 57, and more accurately, the emitting surface is made to face the base end in some degree. The ultrasonic array element 53 has a sound medium 59, such as silicone rubber, provided on the front side of the emitting surface. The sound medium 59 has a function of efficiently transmitting the ultrasound emitted from the ultrasonic array element 53 to the heart E at the time when the device head portion 51 is fixed in contact with the surface of the heart E.

The ultrasonic array element 53 is also connected to an electric line 52 for signal transmission and reception. The electric line 52 is connected to a connector (illustration omitted) of the grip portion 31 through an exclusive hole (illustration omitted) provided inside the device head portion 51 and the shaft portion 41. Through the connector, the electric line 52 is connected to the ultrasound observation apparatus 61.

As illustrated in FIGS. 5(b) and 5(c), the case 57 includes a pair of protruded guiding projection portions 57a provided on both sides of an internal surface across the permanent magnet 55. The guiding projection portions 57a extend along the axis direction.

The permanent magnet 55 is placed in close proximity to the ultrasonic array element 53 at a position closer to the base end side of the case 57 than the ultrasonic array element 53. The permanent magnet 55 has a pair of guiding recess portions 55a which are recessed in accordance with the guiding projection portions 57a of the case 57. The permanent magnet 55 is regulated by the guiding recess portions 55a and the guiding projection portions 57a of the case 57 so as to be moved in the axis direction of the case 57 in response to the operation of the magnet drive knob 35 of the grip portion 31.

The permanent magnet 55 is fixed to a spring 54 that is provided with a tension to press the permanent magnet 55 toward the distal end of the case 57, and to a drive wire 56 to pull the permanent magnet 55. The drive wire 56 is connected to a part of a member (not illustrate) that operates in conjunction with the lever 33 of the grip portion 31 through a wire hole (illustration omitted) provided inside the device head portion 51 and the shaft portion 41.

The permanent magnet 55 is placed so that the surface facing in a direction of ultrasound radiation by the ultrasonic array element 53, i.e., the surface that is made to face the surface of the heart E, has a polarity different from the polarity of the magnetism of the magnetic head 25 of the guidewire 12 that is directed outward in the radial direction of the magnetic head 25. In the present embodiment, the surface of the permanent magnet 55 that faces the surface of the heart E has the S pole. Accordingly, magnetic attraction force can be made to act between the permanent magnet 55 of the device head portion 51 and the magnetic head 25 of the guidewire 12.

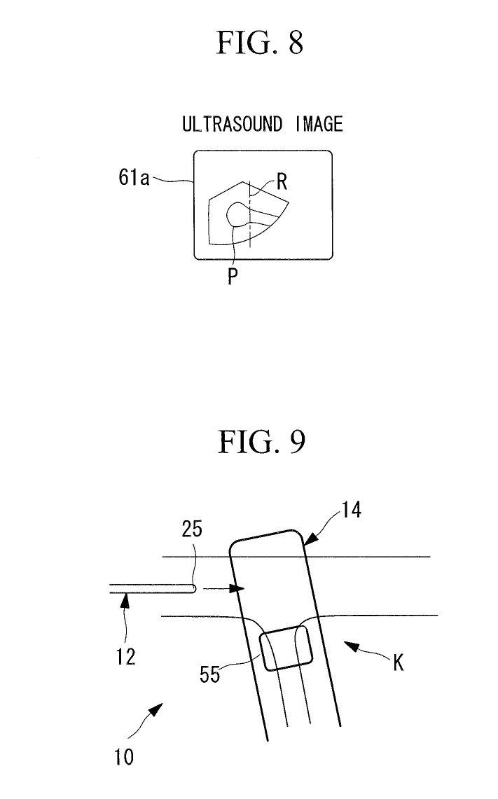

The ultrasound observation apparatus 61 can indicate on the monitor 61a an ultrasound tomographic image of cardiac muscle tissues acquired by the ultrasonic array element 53 by using publicly known ultrasound techniques (such as a sector scanning method). The monitor 61a of the ultrasound observation apparatus 61 can display a line-shaped marker R (see FIG. 8), which indicates the position of the permanent magnet 55 of the guiding device 14, on the ultrasound image. This enables an operator to recognize at a glance which portion of the epicardium the permanent magnet 55 is positioned at in the ultrasound image.

A description is now given of the functions of the thus-configured guiding-type medical system 10 according to the present embodiment.

For example, in treatment of coronary artery disorders, the guidewire 12 of the guiding-type medical system 10 according to the present embodiment is detained in a coronary artery blood vessel. For example, a method by Sosa (reference: Sosa E et al., Nonsurgical transthoracic epicardial catheter ablation to treat recurrent ventricular tachycardia occurring late after myocardial infarction, J Am Coll Cardiol 2000; 35:1442-1449) is used to detain two sheaths 69 with a steering mechanism, which access into the pericardial cavity G, below a xiphisternum of the patient as illustrated in FIG. 1. Through the respective sheaths 69, the endoscope 63 and the guiding device 14 are inserted into the pericardial cavity G.

Next, the guidewire 12 is inserted into the coronary artery C through a femoral artery via the introducer 67. By operating the grip portion 31, the guidewire 12 is moved toward a desired blood vessel route. As the guidewire 12 is moved into more intricately-structured blood vessels, such as blood vessel branch parts, it becomes difficult to move the guidewire 12 toward a desired blood vessel route only by the operation of the grip portion 31.

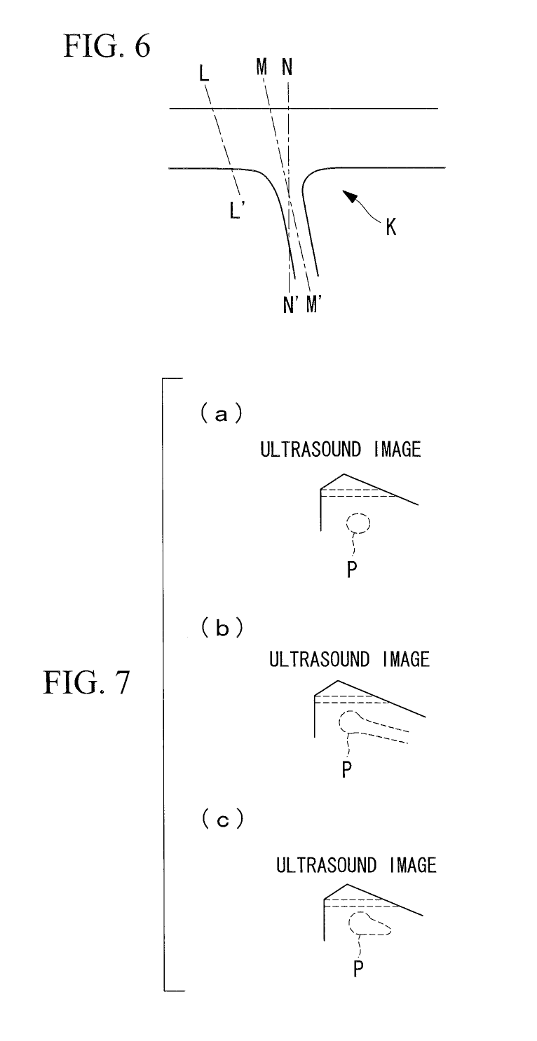

Accordingly, the endoscope 63 inserted into the pericardial cavity G is used to confirm an approximate position of a blood vessel branch part K (see FIG. 6) at which the blood vessel route to insert the guidewire 12 is bifurcated. In the vicinity of the confirmed branch part K, the device head portion 51 of the guiding device 14 is temporarily placed by using the sheaths 69 and the steering mechanism (such as the curving mechanism 45) of the guiding device 14.

Then, the ultrasonic array element 53 included in the device head portion 51 of the guiding device 14 is operated to acquire an ultrasound image of the blood vessel while an ultrasound scan is performed on the blood vessel. The acquired ultrasound image is displayed on the monitor 61a of the ultrasound observation apparatus 61. Depending on the positions of the blood vessel to be scanned with ultrasound, different tomographic images are acquired.

For example, when an ultrasound scan is performed at a position other than the blood vessel branch part K in a direction intersecting an extending direction of a blood vessel, such as an L-L′ position of FIG. 6, a tomographic image P of a substantially circular or elliptical blood vessel is generally included in an obtained ultrasound image as illustrated in FIG. 7(a). When an ultrasound scan is performed at a position such as an M-M′ position of FIG. 6 in a direction in which a bifurcated blood vessel extends past the blood vessel branch part K, a long axis image (tomographic image P) representing the bifurcated blood vessel is indicated in an ultrasound image as illustrated in FIG. 7(b). When an ultrasound scan is performed at a position such as an N-N′ position of FIG. 6 in the direction in which the bifurcated blood vessel extends past the blood vessel branch part K, a tomographic image P of the blood vessel which gradually disappears toward the extending direction of the bifurcated blood vessel is indicated in an ultrasound image as illustrated in a FIG. 7(c).

Accordingly, when the guidewire 12 inserted into a blood vessel comes closer to the blood vessel branch part K, first an operator places the device head portion 51 of the guiding device 14 so that an ultrasound scan is performed at a position such as the M-M′ position of FIG. 6 in the direction in which a desired bifurcated blood vessel extends past the blood vessel branch part K. Then, as illustrated in FIGS. 7(b) and 8, an ultrasound image including a long axis image of a desired blood vessel route is acquired by the ultrasonic array element 53. The position of the device head portion 51 is finely adjusted so that the marker R representing the position of the permanent magnet 55 is aligned with an inlet of the blood vessel route at the blood vessel branch part K on the ultrasound image.

In this state, the guidewire 12 is moved toward the blood vessel branch part K as illustrated in FIG. 9. At this point, the guidewire 12 is not indicated in the ultrasound image.

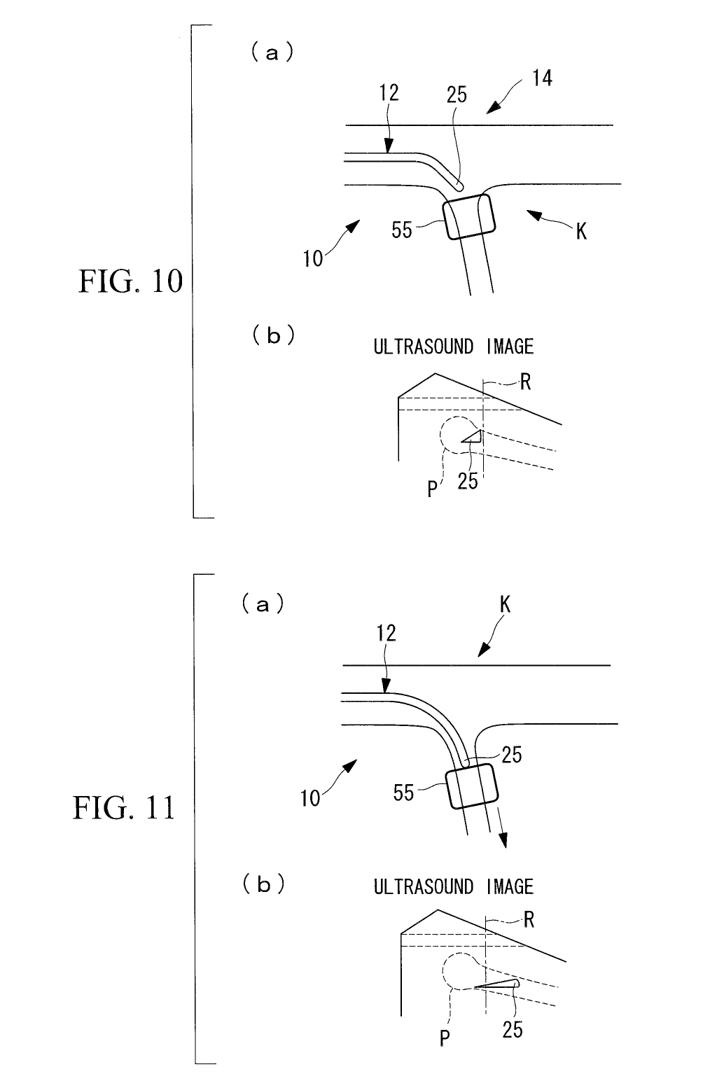

In this state, the distal end of the guidewire 12 is moved closer to the device head portion 51 of the guiding device 14. Consequently, as illustrated in FIG. 10(a), the permanent magnet 55 of the guiding device 14 causes magnetic attraction force to act on the magnetic head 25 of the guidewire 12, so that the magnetic head 25 is attracted to the permanent magnet 55 of the guiding device 14.

In this state, the distal end of the guidewire 12 being in proximity of the magnetic head 25 of the guiding device 14 is irradiated with the ultrasound from the ultrasonic array element 53. As a result, as illustrated in FIG. 10(b), a tomographic image of the distal end of the guidewire 12 is indicated in an ultrasound image. The image can ensure that the magnetic head 25 of the guidewire 12 is attracted to the permanent magnet 55 of the guiding device 14 and is thereby moved toward the desired blood vessel route.

In this case, the projections and indentations provided on the outer surface of the magnetic head 25 can reflect the ultrasound from the ultrasonic array element 53 in multiple directions. As a result, some reflected light can reliably be made incident into the ultrasonic array element 53, and the magnetic head 25 of the guidewire 12 can efficiently be included in an ultrasound image.

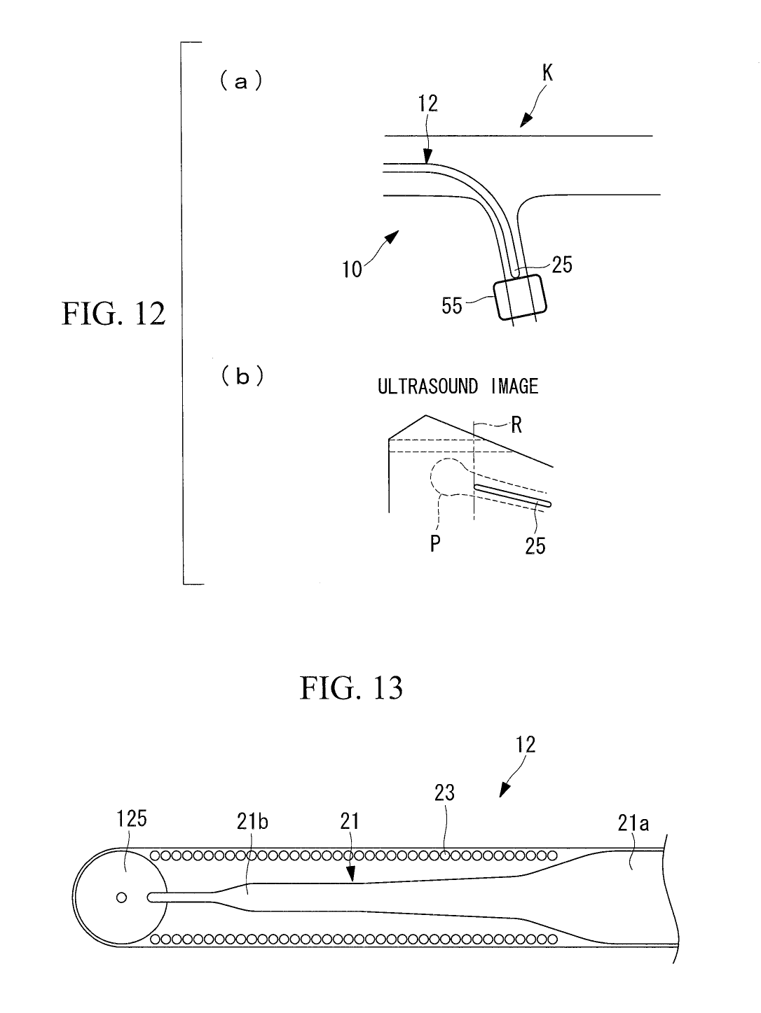

Next, as illustrated in FIGS. 11(a) and 12(a), the magnet drive knob 35 of the grip portion 31 is operated to move the permanent magnet of the guiding device 14 along the extending direction of the blood vessel to which the guidewire 12 is inserted, at a speed substantially identical to the moving speed of the guidewire 12. In this case, as illustrated in FIGS. 11(b) and 12(b), the guidewire 12 drawn into the desired blood vessel route in synchronization with the movement of the permanent magnet 55 of the guiding device 14 is indicated in an ultrasound image. By referring to the image, the guidewire 12 can be inserted into the desired blood vessel route.

As described in the foregoing, according to the guiding-type medical system 10 in the present embodiment, a desired blood vessel route is indicated in an ultrasound image by the ultrasonic array element 53. By referring to the image, the permanent magnet 55 of the guiding device 14 can be placed in a desired position. For example, in some cases, it is difficult to visually recognize a target blood vessel with an image obtained by the endoscope 63 inserted into the pericardial cavity G due to such factors as a thick fat layer. Even in such cases, the ultrasonic array element 53 of the guiding device 14 makes it possible to accurately position the permanent magnet 55. Since the ultrasound scanning direction by the ultrasonic array element 53 of the guiding device 14 is aligned with the moving direction of the permanent magnet 55, the magnet 55 of the guiding device 14 is linearly moved with the magnetic attraction force being made to act on the magnetic head 25 of the guidewire 12 while an ultrasound scan is performed on a desired blood vessel in the extending direction of the blood vessel. As a result, the distal end of the guidewire 12 can easily be guided along the desired blood vessel.

The present embodiment can be modified as shown below.

In the present embodiment, although the magnetic head 25 made of a magnetic material has been described as an example of the magnetic member of the medical appliance, a hemispherical or cannonball-shaped magnet having a hemispherical surface that is magnetized to the N pole may be used as the magnetic member of the medical appliance in a first modified example.

By using a magnet as the magnetic member of the medical appliance, intense interaction (magnetic attraction force) with the permanent magnet 55 of the guiding device 14 is generated. Therefore, the guiding force by the permanent magnet 55 of the guiding device 14 can be intensified. In this case, in order to shift the effect of the opposite S pole to the base end side of the guidewire 12, the shaft 21 and/or the coil 23 of the guidewire 12 may be formed of magnetic materials or may be formed from a plurality of magnets.

In the first modified example, as illustrated in FIG. 13, a magnet 125 having a generally spherical shape and having polarity in a radial direction may be adopted as the magnetic member of the medical appliance. In this case, for example, the surface of the magnet 125 may be magnetized to the N pole.

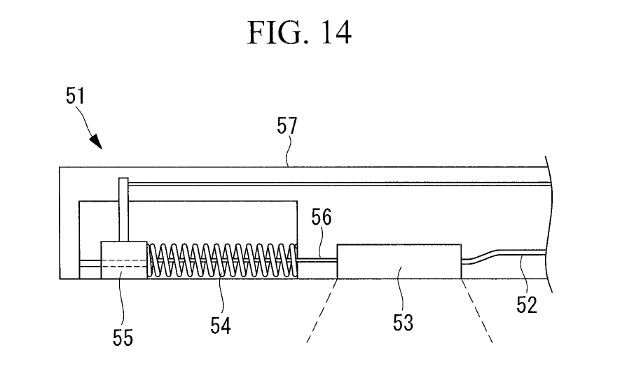

In the present embodiment, the ultrasonic array element 53 is placed at the distal end of the device head portion 51. However, in a second modified example, the ultrasonic array element 53 may be placed closer to the base end side than the permanent magnet 55 in the device head portion 51 as illustrated in FIG. 14. In some cases, the distal end of the device head portion 51 is positioned in an apex cordis direction depending on the placement of the shaft portion 41 of the guiding device 14 inside the pericardial cavity G. The configuration of this modified example is effective for such cases.

### Second Embodiment

Now, a guiding-type medical system in the second embodiment of the present invention is described.

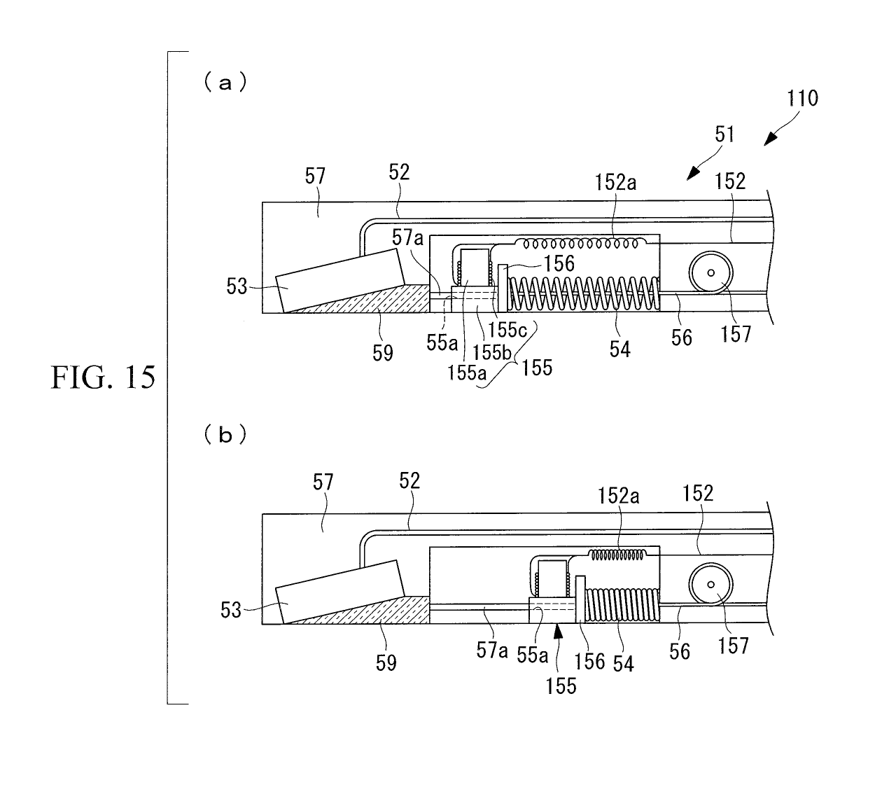

A guiding-type medical system 110 according to the present embodiment is different from the first embodiment in the following point. That is, as illustrated in FIGS. 15(a) and 15(b), the guiding device 14 includes an electromagnet 155 in place of the permanent magnet 55, and the grip portion 31 includes a connector (illustration omitted) which electrically connects the electric line 52 from the electromagnet 155 and the power source (illustration omitted).

Hereinafter, component members common in configuration with the guiding-type medical system 10 according to the first embodiment are designated by common reference numerals to omit description thereof.

The electromagnet 155 includes a columnar magnetic core material 155a, a square plate-like magnetic base 155b joined to a part of the bottom of the magnetic core material 155a, and a coil 155c wound around the magnetic core material 155a.

The magnetic base 155b has a pair of guiding recess portions 155a which are recessed in accordance with the guiding projection portions 57a of the case 57. The electromagnet 155 is regulated by the guiding recess portions 155a and the guiding projection portions 57a of the case 57 so as to be movable in the axis direction of the case 57.

Both ends of the coil 155c are electrically joined to an electric line 152. The electric line 152 has an electric line coil portion 152a spirally wound so as to have a margin in its length in order to prevent disconnection by movement of the electromagnet 155 inside the device head portion 51. The electric line 152 extends through the shaft portion 41 so as to be connected to the power source through the connector of the grip portion 31. For example, in the case of guiding the guidewire 12 including the magnet placed to have an N-pole distal end, the power source supplies the current which makes the surface of the electromagnet 155 that comes into contact with tissues have the S pole.

The magnetic base 155b is fixed to a spring supporting plate 156.

The spring 54 loaded with a tension to push the electromagnet 155 toward the distal end of the magnetic head 25 is inserted and fixed to the spring supporting plate 156. The spring supporting plate 156 is also connected to the drive wire 56 for driving the electromagnet. The other end of the drive wire 56 extends through the hollow of the spring 54 so as to be connected, through a pulley 157, to a member (illustration omitted) which operates in conjunction with the magnet drive knob 35 of the grip portion 31.

The thus-configured guiding-type medical system 110 according to the present embodiment can pass the current to the electromagnet 155 to generate a magnetic field only in the case of guiding the guidewire 12 inserted into a blood vessel. In any other occasions, the guiding-type medical system 110 can block current supply to the electromagnet 155 so as to prevent generation of the magnetic field. Therefore, in the state where the current supply to the electromagnet 155 is stopped, the guiding-type medical system 110 can also be used for acquiring ultrasound images of other portions of the heart where an influence of the magnetic field by the ultrasonic array element 53 is undesirable.

### Third Embodiment

Now, a guiding-type medical system in the third embodiment of the present invention is described.

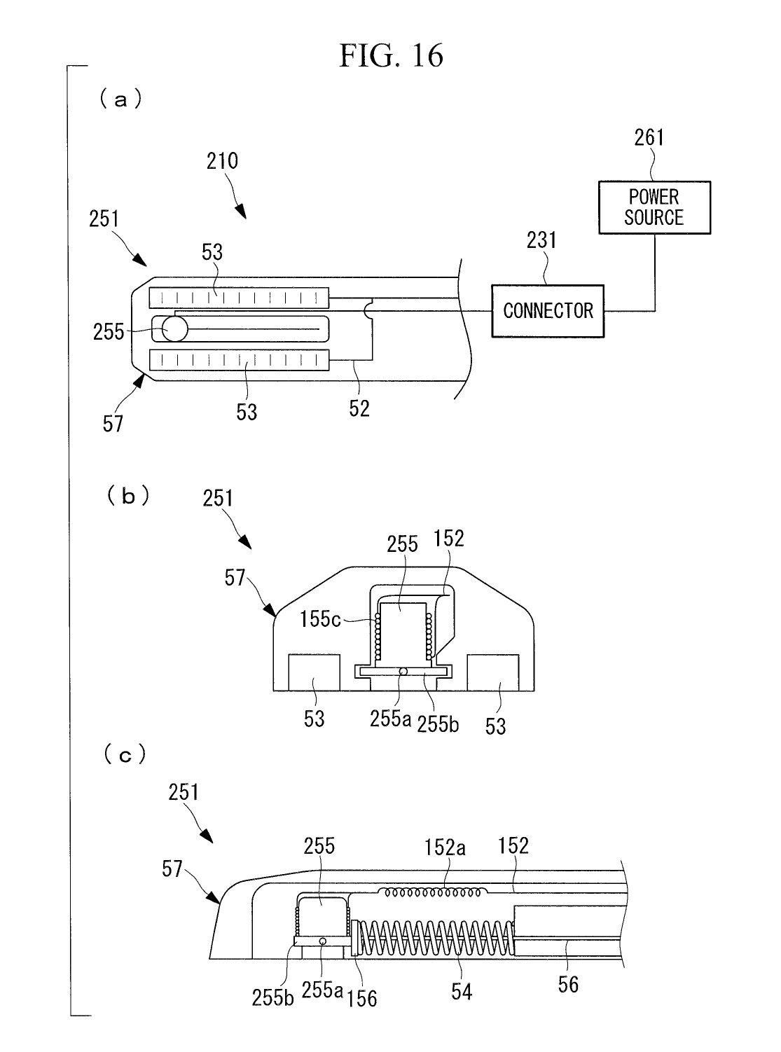

As illustrated in FIGS. 16(a), 16(b), and 16(c), a guiding-type medical system 210 according to the present embodiment is different from the first and second embodiments in the configuration of a device head portion 251 of the guiding device 14.

Hereinafter, component members common in configuration with those of the guiding-type medical system 10 according to the first embodiment and the guiding-type medical system 110 according to the second embodiment are designated by common reference numerals to omit description thereof.

The device head portion 251 according to the present embodiment includes an electromagnet 255 in place of the permanent magnet 55.

The electromagnet 255, which is formed into a cylindrical shape, is bonded and fixed to a plate-like guide plate 255b with a pin-shaped guide plate fixing device 255a. The guide plate 255b is engaged with a guiding recess portion 257a provided in the internal surface of the case 57 in the state freely movable in a long axis direction of the case 57 in a space inside the case 57 of the device head portion 251. Consequently, the electromagnet 155 is regulated by the guide plate 255b and the guiding recess portion 257a of the case 57 so as to be movable in the axis direction of the case 57.

The guide plate 255b is also connected to the drive wire 56 and the spring 54.

The drive wire 56 is connected to a member which operates in conjunction with the magnet drive knob 35 of the grip portion 31 through a drive wire whole (illustration omitted) provided in the shaft portion 41. The other end of the spring 54 is loaded with a tension to push the electromagnet 155 toward the distal end of the case.

Accordingly, when the magnet drive knob is operated, the drive wire 56 is pulled so that the electromagnet 255 moves to the base end side of the case 57. When the magnet drive knob is released, the drive wire 56 is loosened and the electromagnet 255 returns to an initial position of the case 57 due to restoring force of the spring 54.

In the present embodiment, one ultrasonic array element 53 is placed on both sides of the electromagnet 255 along the moving direction of the electromagnet 255. These ultrasonic array elements 53 are electrically connected to each other to constitute one element, which is connected to the ultrasound observation apparatus 61 through a connector 231 of the grip portion 31.

The thus-configured guiding-type medical system 210 is used by the same method as in the first embodiment except in the following point. That is, when the guidewire 12 is guided by the guiding device 14, the current is supplied to the electromagnet 255 from a power source 261 so that the surface of the electromagnet 255 that comes into contact with tissues is made to have S-pole magnetism.

As described above, in the guiding-type medical system 210 according to the present embodiment, the ultrasonic array elements 53 are symmetrically placed on both the sides of the electromagnet 255, so that the size of the long axis direction of the case 57 can be reduced as compared with the case where the ultrasonic array element is placed closer to the base end side or the distal end side of the case 57 than the electromagnet 255.

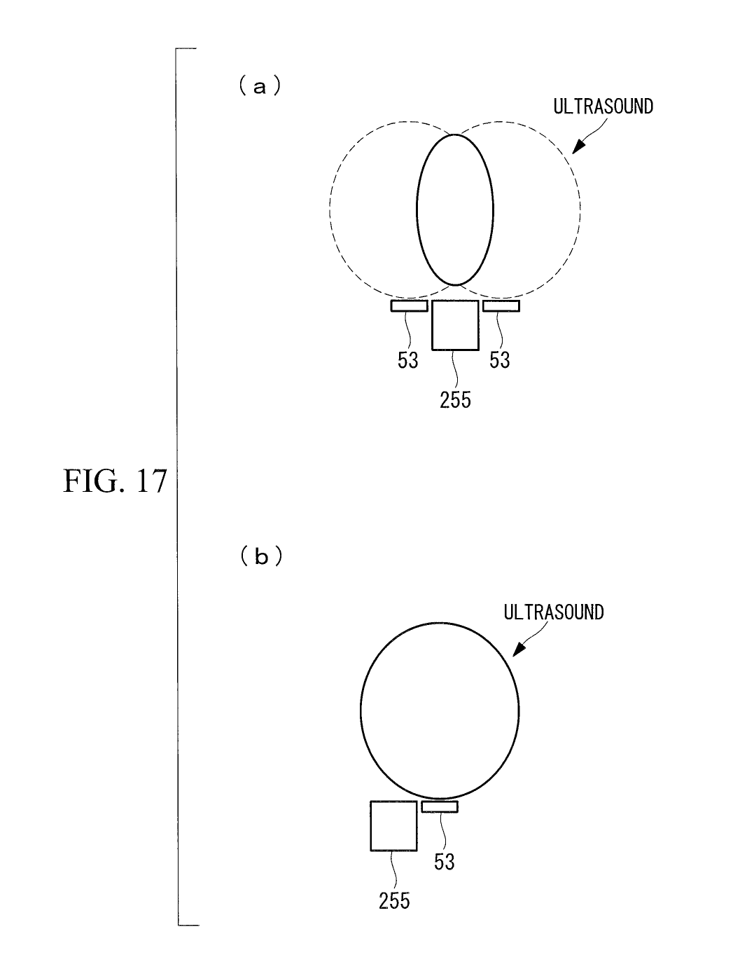

With such configuration, ultrasound is emitted to the area directly below the electromagnet 255, so that a tomographic image of intracardiac tissues placed directly below the electromagnet 255 can be acquired as illustrated in FIG. 17(a). Therefore, as compared with the case where an ultrasound image is acquired by the ultrasonic array element 53 placed only on one side of the electromagnet 255 as illustrated in FIG. 17(b), it becomes possible to reduce misalignment of the position of the electromagnet 255 with the ultrasound image in a width direction. It also becomes possible to facilitate more accurate adjustment in placement of the desired blood vessel route and the electromagnet 255. Therefore, the guidewire 12 can more accurately be guided.

In the foregoing, the embodiments of the present invention have been explained in full detail with reference to the accompanying drawings. However, the specific configuration of the invention is not limited to the embodiments disclosed, and various modifications and variation in design which come within the scope of the present invention are intended to be embraced therein. For example, the present invention is not limited to each of the above-stated embodiments and their modifications, and it is possible to apply the present invention to those configured by appropriately combining these embodiments and their modifications without being particularly limited thereby. In each of the embodiments, an example of using the ultrasonic array element 53 as the ultrasound probe has been described. However, instead of the ultrasonic array element 53, a single version vibrator that performs a mechanical scan may be adopted as the ultrasound probe.

In each of the embodiments, the curving mechanism 45 that bends the shaft portion 41 only in a horizontal direction with respect to the surface of the heart E is adopted as a steering mechanism of the shaft portion 41 to simplify the device configuration. For vertical operation, the main body of the guiding device 14 is curved in advance in the direction of the surface that is fixed in contact with the surface of the heart E. However, a publicly known mechanism that enables bending operation in two directions may be adopted.

In the first and second embodiments, the ultrasonic array element 53 is placed only on one side of the permanent magnet 55 or the electromagnet 155. Instead of this configuration, the ultrasonic array element 53 may be placed on both the sides of the permanent magnet 55 or the electromagnet 155 as in the third embodiment.

According to above embodiments, the insertion portion of the guiding device is inserted into the body and the ultrasound probe is used to perform an ultrasound scan on a blood vessel. As a result, an ultrasound tomographic image of a region including the blood vessel can be acquired, so that the position of the blood vessel can be ascertained. The medical appliance is inserted into the blood vessel so that the distal end of the medical appliance is made to appear on an ultrasound tomographic image of the region including the blood vessel. Consequently, the position of the medical appliance inside the blood vessel can be ascertained. Therefore, while the distal end of the medical appliance is being checked with the ultrasound tomographic image of the region including the blood vessel acquired by the ultrasound probe, the magnet of the guiding device is moved with the magnetic attraction force being made to act on the magnetic member of the medical appliance. As a result, it becomes possible to attract and thereby move the distal end of the medical appliance in the blood vessel.

In this case, an ultrasound scanning direction by the ultrasound probe of the guiding device is aligned with a moving direction of the magnet. Accordingly, the magnet of the guiding device is linearly moved with the magnetic attraction force being made to act on the magnetic member of the medical appliance while an ultrasound scan is performed on a desired blood vessel in an extending direction of the blood vessel. As a result, the distal end of the medical appliance can easily be guided along the desired blood vessel.

In the above embodiments, the magnetic member may be a magnet having a generally spherical shape and having polarity in a radial direction.

Such configuration can reduce an influence of the opposite poles of the magnet of the medical appliance exerted on the guiding operation with use of the magnet of the guiding device.

In the above embodiments, the magnet may be an electromagnet.

With such configuration, stopping power supply to the electromagnet can block magnetic interaction between the insertion portion of the guiding device and the distal end of the medical appliance. Therefore, ultrasound images of the inside of the body can be acquired to observe the state of the inside of the body with use of the ultrasound probe of the guiding device without affecting the medical appliance.

In the above embodiments, the distal end of the medical appliance may be shaped to have projections and indentations on an outer surface thereof.

With such configuration, when the distal end of the medical appliance is irradiated with ultrasound emitted from the ultrasound probe, the ultrasound can be reflected in multiple directions due to the projections and indentations on the outer surface of the distal end. As a consequence, some of the ultrasound reflected on the distal end of the medical appliance can reliably be entered into the ultrasound probe, so that an ultrasound image of the distal end of the medical appliance can efficiently be acquired. This makes it possible to clearly recognize the positional relationship between the blood vessel and the distal end of the medical appliance shown on the ultrasound image.

In the above embodiments, the ultrasound probe may be placed at both sides of the magnet of the insertion portion.

Such configuration can provide an ultrasound image bilaterally symmetrical with the magnet of the insertion portion as a center that is acquired with the ultrasound emitted from two ultrasound probes. Consequently, the magnet of the guiding device can accurately be moved and placed close to the desired blood vessel displayed on an ultrasound image.

The present invention provides the advantage that the medical appliance can easily be inserted to a desired blood vessel route.

### Appended Feature 1

A method for guiding a medical appliance, including: a temporary placement step of using an endoscope that is inserted into a pericardial cavity to confirm a position of a desired blood vessel to which a medical appliance is desired to be inserted, and temporarily placing a guiding device in a vicinity of the desired blood vessel; an image acquisition step of acquiring an ultrasound image of the desired blood vessel while an ultrasound scan is performed on the desired blood vessel by an ultrasonic array element of the guiding device; an adjustment step of adjusting a position of the guiding device based on the ultrasound image of the blood vessel acquired by the image acquisition step so as to align an extending direction of the desired blood vessel with a scanning direction of the ultrasound; and a moving step of linearly moving a magnet of the guiding device with magnetic attraction force being made to act on a magnetic member provided at a distal end of the medical appliance while the ultrasound scan is performed on the desired blood vessel in the extending direction of the blood vessel.

By implementing these steps, the guiding device can easily and precisely be placed on a desired blood vessel by visually confirming the ultrasound image without using an X-ray radioscopy. The magnet of the guiding device is linearly moved with the magnetic attraction force being made to act on the magnetic member of the medical appliance while the ultrasound scan is performed by the ultrasonic array element of the guiding device on the desired blood vessel in the extending direction of the blood vessel. As a result, the medical appliance is attracted to the guiding device and is thereby moved while the position is checked with the ultrasound image. This makes it possible to easily guide the medical appliance along the desired blood vessel.

### REFERENCE SIGNS LIST

10,110,210 Guiding-type medical system
12 Guidewire (medical appliance)
14 Guiding device
25 Magnetic head (magnetic member)
51,251 Device head portion (insertion portion)
53 Ultrasonic array element (ultrasound probe)
55 Permanent magnet (magnet)
125 Magnet (magnetic member)
155,255 Electromagnet (magnet)

1. A guiding-type medical system, comprising:
a cylindrical or linear medical appliance made of an elastic body that can be inserted into a blood vessel; and
a guiding device having an insertion portion that can be inserted into a body to guide the medical appliance inserted into the blood vessel, wherein
the insertion portion includes:
an ultrasound probe that can perform an ultrasound scan along one plane to acquire an ultrasound image; and
a magnet linearly movable in a direction along the one plane, and
at a distal end of the medical appliance, a magnetic member made of a magnetic material on which magnetic attraction force induced by the magnet is made to act is provided.

2. The guiding-type medical system according to claim 1, wherein the magnetic member is a magnet having a generally spherical shape and having polarity in a radial direction.

3. The guiding-type medical system according to claim 1, wherein the magnet is an electromagnet.

4. The guiding-type medical system according to claim 1, wherein the distal end of the medical appliance is shaped to have projections and indentations on an outer surface thereof.

5. The guiding-type medical system according to claim 1, wherein the ultrasound probe is placed at both sides of the magnet of the insertion portion.
